(19) United States
(12) Patent Application Publication
(10) Pub. No.: US 2009/0324664 A1
(43) Pub. Date: Dec. 31, 2009

(54) SPRAY-ON, NON-WOVEN FABRIC SYSTEM AND MULTILAYER TOPICAL COVERING

(76) Inventors: John T. Ulman, Woodbridge, NJ (US); Maria Aparecida de Aledo, Princeton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/164,443

(22) Filed: Jun. 30, 2008

(51) Int. Cl. A61K 9/00; A61F 13/00; A61K 33/26; A61K 33/30; A61K 31/135

(52) U.S. Cl. 424/402; 602/48; 424/646; 424/642

(57) ABSTRACT

The invention is directed to a spray-on, multilayer, non-woven fabric system and multilayer topical coverings suitable for reducing or alleviating a discomforting condition of the skin and/or an area underlying the skin when applied thereto, where the fabric system includes a first spray-on composition containing a material suitable for reducing or alleviating a discomforting condition of the skin and/or an area underlying the skin when applied thereto, and a second spray-on composition, different from the first spray-on composition for covering the first composition, which includes fibers, a volatile carrier, and a binding agent, and where the multilayer topical covering includes a first layer formed from the first spray-on composition and a second layer covering the first layer, the second layer formed from the second spray-on composition.

### FIELD OF THE INVENTION

This invention relates to spray-on, non-woven fabrics systems and to multilayer topical coverings provided by such systems.

### BACKGROUND OF THE INVENTION

Fabrics are utilized in a wide variety of applications and industries, including for instance the cleaning and cosmetic industries where fabrics are used as applicators for various active agents. Fabrics are also useful in a number of medical applications. The fabrics may be woven or non-woven. Fabrics are known to be used in adhesive bandages as wound contacting pads.

World Patent Application WO 03/104540 discloses a composition comprising short fibers, a binder and a diluent, which can be sprayed onto a supporting surface to produce a non-woven fabric. The examples describe a single-phase composition prepared with ethyl acetate, acetone or methanol as a diluent. Other diluents mentioned in the description are water, alcohols, ketones and esters having between 1 and 12 carbon atoms. Substantially all of the diluent evaporates before the composition reaches the supporting surface and so the fabric formed comprises the fibers and binder with any optional additives, but without any diluent.

World Patent Application WO 2007/000598 discloses an improvement to the spray-on fabric technology wherein 2 different solvents are utilized, resulting in a fabric that contains a liquid. There is a need for spray-on compositions that are useful for promoting wound healing as well as protecting the wound.

### SUMMARY OF THE INVENTION

The present invention is directed to a spray-on, non-woven fabric system for reducing or alleviating a discomforting condition of the skin and/or an area underlying the skin, including a first spray-on composition including a material suitable for reducing or alleviating a discomforting condition of the skin and/or an area underlying the skin; and a second spray-on composition, different than the first spray-on composition, for covering the first spray-on composition when applied thereto, the second composition including fibers, a binder for the fibers and a volatile carrier for solubilizing the binder in the second composition, and to multilayer topical coverings provided by the spray-on, non-woven fabric systems.

### DETAILED DESCRIPTION OF THE INVENTION

In certain embodiments, when the spray-on system is topically applied to an area of skin, the first spray-on composition is sprayed on and contacts the skin directly and forms a first layer that serves to reduce or alleviate a discomforting condition of the skin and/or an area underlying the skin, for example the muscles. In embodiments where a wound may be present in the area of skin to which the spray-on system is employed, the first layer may utilize a non-volatile carrier and/or may contain an active material to promote wound healing when in contact with the wound. The second spray-on composition, which is different than the first spray-on composition, is then applied over the first layer formed by the first composition and serves as a protective cover over the first layer. As such, a multilayer topical covering is provided that reduces or alleviates a discomforting condition of the skin or the area of the body to which it is applied.

In other embodiments of the invention, a third spray-on composition may be employed to provide a third layer disposed between the first and second layers, or between the skin and the first layer. The material for reducing or alleviating the discomforting condition may be contained within one or both of the first and third layers, depending on the particular application.

Non-volatile carriers that may be employed may be selected from the group consisting of hydrocolloid adhesives, hydrogels and ointments. As is known in the art, hydrocolloid adhesives typically include a hydrophobic adhesive and a fluid absorbing material. The hydrophobic adhesive may be selected from the group consisting of silicones, polyisobutylenes and derivatives thereof, acrylics and natural rubbers. The fluid absorbing material may be selected from the group consisting of methylcellulose, hydroxyethyl methylcellulose, hydroxypropyl methylcellulose, ethyl hydroxyethyl cellulose, hydroxyethylcellulose, hydroxypropylcellulose, carboxymethlcellulose, polyvinyl alcohol, polyvinylpyrrolidone, polyethylene oxide, gum arabic, gum karaya, gum tragacanth, guar gum, gum benzoin, alginic acid and salts of any of the above. The fluid absorbing material may be utilized in amounts up to 40 percent, for example up to 20 percent or 10 percent, or up to 5 percent, by weight of the adhesive composition.

The relative volatilities of the respective carriers reflect the purpose of each of these components. The volatile carrier is a liquid in the composition before spraying, but evaporates upon spraying. The volatile carrier, however, may have a boiling point below room temperature and, to obtain liquid conditions, the composition can be stored at a high pressure. For example, the volatile carrier can be a propellant such as dimethyl ether. In such a case, the composition is stored in a pressurized container such as a metal cylinder colloquially known as an “aerosol can” under a pressure of above 1 and less than 20 atmospheres.

The purpose of the volatile carrier is to solubilize the binder in the spray-on composition so that the composition can be sprayed. If the binder were not soluble in the composition, there would be problems with forming a coherent fabric from the fibers and spraying of the composition. Upon spraying, the volatile carrier evaporates once the composition has been formed into a spray, before the spray reaches the surface of the wound and the fabric is formed thereon. Once the volatile carrier has evaporated, the binder is left in solid form to bind together the fibers to form a fabric covering the skin surface.

The volatile carrier is normally an organic solvent for example a C₂ to C₁₂ ether, such as diethyl ether, a C₂ to C₁₂ alcohol, a C₂ to C₁₂ ketone, a C₅ to C₁₂ hydrocarbon, or a C₂ to C₁₂ ester. The volatile carrier may also be a propellant such as dimethyl ether or liquefied petroleum gas, which is a commercial mixture of propane and butane.

It is advantageous to have a propellant as the volatile carrier, as the volatile carrier will have a further function of propelling the spray. When the composition comprises a propellant, the composition should be stored in a pressurized container. When an opening is made in the container, usually at a nozzle for spraying, the propellant, with the rest of the composition, leaves the container in a form of a spray as a result of the energy stored in the pressurized propellant.

Where applicable, the purpose of the non-volatile carrier is to maintain moisture in the first layer upon application to the skin, thus aiding in healing of the wound if present. Therefore, the non-volatile carrier should not substantially evaporate during spraying and should remain liquid on the skin or as incorporated into the fabric. The non-volatile carrier is a liquid under ambient conditions (20° C. and 1 atmosphere) so giving the fabric a “wet” character.

The non-volatile carrier may be selected from the group consisting of water, ketones, alcohols, esters and ethers. Preferably, the non-volatile carrier is selected from the group consisting of water, acetone, ethanol, polyhydric alcohols and glycol ethers. Suitable polyhydric alcohols include ethylene glycol, propylene glycol, isoprene glycol, dipropylene glycol, glycerol, 1,3-butylene glycol, sorbitol, diethylene glycol monoethyl ether, and mixtures thereof. Preferably, the non-volatile carrier comprises water. In preferred embodiments, at least 30%, preferably at least 50%, of the non-volatile carrier is water.

The volatile and non-volatile carriers may or may not be miscible with each other. Where they are not miscible, the composition comprises an emulsion. An emulsifier can be included to facilitate formation of an emulsion. The emulsifier is preferably designed to produce an emulsion of non-volatile carrier in volatile carrier. Surfactants are suitable emulsifiers, for example Span 85 or Tween 20. Mixtures of surfactants may also be used. Having an emulsion of non-volatile carrier in volatile carrier allows the volatile carrier to evaporate easily on spraying, leaving the non-volatile carrier to be incorporated into the fabric.

One requirement of the composition is the relative boiling points of the carriers; so in any particular composition, the combination of carriers is important. Therefore, although acetone, for example, could potentially be a volatile carrier in one composition and a non-volatile carrier in another composition, in any single composition two different liquids are needed.

The volatile carrier may comprise between about 10% and about 80% by weight of the composition based on the total weight, for example between about 20% and about 50%, while the non-volatile carrier may comprise between about 5% and about 60% by weight of the composition based on total weight, for example between about 10% and about 50%.

The second spray-on composition forms a dry fabric, preventing clothing or the exterior of the fabric from being wet or contacting the first layer. The second composition contains fibers, a binding agent and a volatile carrier, each as described above. The amount of volatile solvent is increased accordingly to account for the lack of non-volatile solvent and/or active material. In certain embodiments the second composition is essentially free of non-volatile carriers, for example less than about 2% by weight of the composition. In other embodiments, the second spray-on composition is free of non-volatile carriers.

Both first and second spray-on compositions, and third composition when used, may comprise a binding agent, or “binder”, that is solid at ambient temperature, but is soluble in the volatile carrier. Upon evaporation of the volatile carrier after spraying, the binder acts to bind the fibers into a non-woven fabric covering the skin area, in the case of the first layer, and covering the first layer, in the case of the second layer. Hence the binder and the volatile carrier are matched so that they are complementary. The binder should be substantially insoluble in the non-volatile carrier. The requirement for binder to be soluble in the volatile carrier but not soluble in the non-volatile carrier applies under circumstances where both the volatile carrier and non-volatile carrier are liquid. For the volatile carrier, this may not be at normal atmospheric pressure, i.e. 1 atmosphere, in particular where the volatile carrier is a propellant. In the composition, the binder is dissolved in the volatile carrier. Therefore, the binder needs to be soluble in the volatile carrier at the relative levels of these compounds present in the composition.

A binder should be selected so that it is soluble in the volatile carrier, preferably an organic volatile solvent, but not soluble in the non-volatile carrier, preferably a polar compound. The binder may be polymeric and can be a homopolymer or a polymer of two or more monomers. The binder is preferably selected from the group consisting of a thermoplastic elastomeric block copolymer such as styrene butadiene, styrene isoprene block copolymers, hydrogenated butadiene styrene, hydrogenated isoprene styrene block copolymers, polyvinyl acetate, polyvinyl butyrate, natural latex and polyvinyl alcohol.

The fabric provided by the present invention is a non-woven textile material, which is often felt-like or fleece-like in texture. Both the first and second compositions, and the third composition where used, and the fabric of the present invention may comprise “fibers” of fabric material which are slender, elongated structures having an aspect ratio of at least 3:1, for example at least 5:1, or at least 10:1. The fibers in the composition must be of a certain minimum length. Generally at least 80% by weight of the fibers have a length of at least 0.02 mm. For example, at least 90%, or 95%, or substantially all of the fibers in the composition may have a length of at least 0.02 mm.

The fibers of the composition should not be too long since a composition comprising long fibers cannot be sprayed easily, because the fibers block a nozzle. Generally, at least 80% by weight of the fibers have a length not more than 10 mm, or not more than 5 mm, for example not more than 1 mm, or not more than 0.5 mm, 0.25 mm, or not more than 0.15 mm. In some embodiments, at least 90%, or at least 95%, or substantially all of the fibers have a length not more than 10 mm, or not more than 5 mm, 1 mm, 0.5 mm, 0.25 mm, or not more than 0.15 mm. In one embodiment substantially all the fibers have a length in the range from about 0.02 to about 0.15 mm.

The fibers should be dispersed in the composition. Where the volatile carrier and non-volatile carrier are not miscible and an emulsion is formed, the fibers may be dispersed in either or both phases. Where there is an emulsion, if hydrophilic fibers are used, the fibers will be preferentially dispersed throughout the non-volatile carrier. If hydrophobic fibers are used, these will be dispersed preferentially throughout the volatile carrier. It is believed that particularly good results may be obtained when a mixture of hydrophilic and hydrophobic fibers are used.

Both synthetic and natural fibers may be used in the composition. The fibers may be natural fibers, such as cotton, to provide a fabric that is similar to cotton wool, or silk to give a smooth cloth. Alternatively, synthetic fibers may be used, such as polypropylene, polyethylene or polyamide, which can give “fleece like” fabrics.

Generally the fibers in the composition are at least 10%, 20%, 30% or 40% polymeric fibers, by total weight of fibers. In a preferred embodiment most of the fibers in the composition are polymeric and organic, even though other fiber types may be used.

Thus preferably at least 50%, 60%, 70% or 80%, especially at least 90%, of the fibers, by weight, are polymeric. Most preferably, only organic, polymeric fibers are used.

For the composition when sprayed to produce a non-woven fabric a sufficient binder to fiber ratio is usually desirable. For example the ratio of binder to fibers is preferably not greater than 5:1, more preferably not greater than 4:1 and even more preferably not greater than 3:1 and is preferably not less than 1:5, more preferably is not less than 1:4 and more preferably is not less than 1:3. Preferably, the ratio of fibers to binder is in the range 5:1 to 1:5, more preferably 3:1 to 1:3, even more preferably 2:1 to 1:2, most preferably 1.5:1 to 1:1.5.

3. The first spray-on composition comprises a material suitable for reducing or alleviating a discomforting condition of the skin or area underlying the skin. For example, the condition may be include, without limitation, inflammation, pain, itch, or stiffness of muscles, which may be caused by injury, infection, etc. The additive may be selected so that it does not evaporate during spraying and can thus become incorporated into the fabric. The material may be, for example, a source of heat, a source of cooling, an antibiotic, an anesthetic, an anti-inflammatory, or an anti-itch composition. The source of heating may be selected from the group consisting of iron oxide powder, capsaicin, zinc-copper and other redox systems, while the source of cooling may be selected from the group consisting of menthol, camphor and hydrogels. The additive may be soluble in the non-volatile carrier. Other materials that could be added to the composition to be sprayed along with the fibers include oils such as citronella, eucalyptus, and neem, etc., perfumes, vitamins, moisturizers, natural antibiotics, proteins, or health & beauty products such as deodorants and antiperspirants, and combinations thereof. The materials may be encapsulated as is known in the art. If the spray-on fabric is required to adhere to a surface, an adhesive agent may also be incorporated. Adhesives used for adhesive bandages are particularly suitable.

Preferably, at least 50%, 75%, 85% or 95% by weight of the total solids in the composition are fibers and binder. The additives must be selected so that the fibers do not aggregate when in the composition. Agents that function as dispersants, such as surfactants may be added to prevent aggregation of fibers in the composition.

For different colors of spray-on fabric, dyed fibers may be used, or small quantities of dye can be added directly to the diluent. Food dyes are particularly suitable, but any dye soluble in the diluent can be used.

The choice of binder and other components also has an effect on the viscosity of the composition. If the viscosity is too high the composition will be difficult to spray.

Preferably, the composition has a viscosity in the range 10 mPas to 100 Pas, more preferably in the range of 1 Pas to 10 Pas. The viscosity of the composition is measured using a concentric cylinder rheometer (Paar Physica Universal Dynamic Spectrometer {UDS] 200) at a temperature of 25° C.

It is not necessary to use only one type of fiber or volatile carrier or non-volatile carrier or binder in the composition. Blends of more than one material for each component may be used. Additionally, if the composition comprises additives, one or more additives may be used in the same composition.

In one embodiment, a first composition containing the material for reducing or alleviating the discomforting condition is stored in one can, and the second composition that does not contain the material is stored in a second can. The consumer may spray apply the first composition to form the first layer, then apply the second composition to form a dry fabric layer. The first layer may comprise a hydrocolloid adhesive or an antibiotic ointment such as Neosporin® or Bacitracin®. It may also comprise a film forming polymer. Suitable film forming polymers include but are not limited to homo or hetero-polymers composed of alkyl acrylates and alkyl methacrylates; where the polymers may be straight chained or branched and the alkyl groups may be butyl, pentyl, hexyl, heptyl, octyl, nonnyl, decyl, undecyl, dodecyl and tridecyl groups.

Multilayer systems of the invention may be applied using an apparatus comprising 2 chambers for containing the respective first and second compositions, which is capable of producing a spray of the compositions from the container. The apparatus contains 2 dip tubes, one for each chamber. The dip tubes are connected to a spray nozzle having 2 spray orifices (one for each composition). The orifices are either side by side or top to bottom to enable spraying of the dry layer on top of the wet or active material layer. The dip tubes are arranged to be curved near the spray nozzle to prevent clogging of the tubes. The compositions in the container may be at a pressure of above 1 and below 20 atmospheres and preferably between 3 and 20 atmospheres, more preferably between or 8 and 15 atmospheres. The volatile carrier and non-volatile carrier should both be liquid under conditions that prevail in the container. Preferably, the container has a nozzle with internal diameter of 0.05 to 2 mm, more preferably 0.1 to 1 mm. As will be understood, if more than 2 layers are used in the invention, the apparatus will be adapted accordingly.

The fibers in the formed fabric are conjugated and are longer than the fibers in the composition. It is believed that the fibers in the fabric are formed from shorter fibers in the composition joined by binder so that they partially overlap. The conjugated fibers are at least 1 mm, preferably 2 mm, more preferably 5 mm.

During the spraying in step b) of the method, at least 90% of the volatile carrier should evaporate before the composition reaches the support surface but less than 50% of the non-volatile carrier should evaporate. During spraying the spray should be fine so that the volatile carrier can evaporate from the composition and the fibers are able to conjugate during spraying.

Preferably, the container has a nozzle with internal diameter of 0.05 to 2 mm, more preferably 0.1 to 1 mm. This provides the fine spray. Preferably, conjugated fibers form during step b) so that the average length of fibers in the formed non-woven fabric is greater than the average length of fibers in the sprayed composition. Preferably, the spray formed in step b) is a fine spray with droplet size of less than 1 mm.

There are interacting factors, which determine the type of fabric produced by spraying the composition of the present invention. Different volatile carriers can have an effect on the resulting fabric as can changes in the fiber and binder contents as well as the fibers and binders used. Changes in the distance from the point of spraying to the surface can lead to differences in the resulting fabric. When the spray point is close to the surface the fabric adheres more closely to the surface. If the spray point is too close to the supporting surface, insufficient propellant evaporates before the spray hits the surface and a non-woven fabric is not produced, but a film rather than a fabric is formed. Preferably, the distance between the spray point and the surface is greater than 10 cm, more preferably greater than 30 cm. It has been found that if the amount of volatile carrier is increased the optimum spraying distance increases. Similarly, as the boiling point of the volatile carrier increases the optimum spraying distance increases. When the spray point is further away from the surface, the non-woven fabric which forms is easily peeled from the surface, so that the fabric remains as a coherent layer.

The velocity of the composition when it leaves the spray point and when it arrives at the surface can also affect the resulting fabric. The methods and compositions of the invention may be used for a wide range of applications, included without limitation, direct spraying onto the human body, e.g. dressings or bandages for wounds or burns; with UV absorbing technology for sunscreens; to areas experiencing muscle pain, or with fragrance for fragrance emitting patches.

The present invention is also useful in medical applications where moist dressings are often needed. An example of such a medical application is the emergency treatment of burns. Compositions of the present invention could be sprayed onto the affected area of skin to provide a wet cooling dressing. Pharmaceutical additives such as drugs can be incorporated into the composition to make the dressing more effective.

The container from which the composition is sprayed is generally sealed and can be sterilized during manufacture, which means that the fabric produced would be sterile, other than the contact it has had with the atmosphere. This makes it highly suitable for use in medical or cosmetic application.

Examples are set forth below to further illustrate the nature of the invention and the manner of carrying it out. However, the invention should not be considered as being limited to the details thereof.

### EXAMPLE 1

### Medicated Pain Relief Patch

A skin adherent patch can be formed by spraying directly on the skin formulation (A) to form an adhesive layer and then formulation (B) on top of (A) to form a protective barrier. The skin contacting layer (A) contains adhesive, fibers, diluent, and menthol (a topical counter-irritant). Layer (A) was intended to provide enough adhesion so that the pain relief patch will conform to the body despite movement. It also delivers 5% menthol upon contact to provide topical pain relief. Layer (B), sprayed on top of layer (A) is a barrier to prevent A from collecting dirt and sticking to other clothing upon application.

| Layer (A) | | |
|---|---|---|
| Polystyrene-b-polyisoproene-b-polystyrene copolymer | 8 | g |
| Pressurized dimethyl ether | 90 | ml |
| Hydrophobic cotton fibers | 4 | g |
| Menthol | 0.63 | g |
| Layer (B) | | |
| Polystyrene-b-polyisoproene-b-polystyrene copolymer | 2 | g |
| Pressurized dimethyl ether | 90 | ml |
| Hydrophobic cotton fibers | 4 | g |
| Polyethylene fibers | 4 | g |

In addition to menthol, the active ingredient or a combination of active ingredients (e.g. capsaicin, camphor, methyl salicylate) in the patch can be used to deliver medications for various local or systemic effects.

Layer (B) may or may not contain fibers depending on the application. Other ingredients that can form a barrier include various commercially available polymers or film formers.

The pressurized composition can be sprayed from a package without the use of a propellant, such as Bag-on-Valve, Bag-in-Can, Power Pouch, or Mixtek co-dispensing packaging technologies.

### EXAMPLE 2

### Heat or Cold Pain Relief Patch

Topical hot or cold therapies are commonly used in pain management. A multilayer sprayable patch can be produced by applying different ingredients. The consumer or patient will have a choice to spray one or another as needed. Layer (C) contacts the skin and Layer (B) is sprayed on top of C.

Layer (C)

| | | |
|---|---|---|
| Polystyrene-b-polyisoproene-b-polystyrene copolymer | 8 | g |
| Pressurized dimethyl ether | 90 | ml |
| Hydrophobic cotton fibers | 4 | g |
| Highly water absorbing hydrogel | 2 | g |
| (e.g. acrylic acid-acrylate copolymers as | | |
| described in US patent # 5073612) | | |

Layer (B) is the same as in example 1. It may or may not contain fibers depending on the application.

Upon air exposure, the highly water absorbing hydrogel absorbs heat from the skin and evaporates the moisture, thus producing a cooling sensation on the skin.

Other metal combinations (e.g. zinc-copper) or metal oxide (e.g. iron oxide) powder can be used in the spray formulation to generate heat in-situ and produce a heating pad as illustrated in Example 3 below.

### EXAMPLE 3

### Heat Generating Pain Relief Patch

In this example, Layer (D) is contacting the skin, Layer (E) is sprayed on top of D, and Layer B on top of E.

| Layer (D) | | |
|---|---|---|
| Polystyrene-b-polyisoproene-b-polystyrene copolymer | 8 | g |
| Pressurized dimethyl ether | 90 | ml |
| Hydrophobic cotton fibers | 4 | g |
| Layer (E) | | |
| Iron oxide powder | 10 | g |
| Diluent | 2 | g |
| Pressurized isobutane | 88 | g |

Layer (B) is the same as in example 1. It may or may not contain fibers depending on the application. As illustrated in this example, multiple layers can be formed to confer different functional properties for the final product.

### EXAMPLE 4

### Wound Care Adhesive Patch

A two layer spray-on bandage can be formed by first spraying a composition that has a higher adhesive and fluid absorbing fabric that is in contact with the wound and then a second, soft, outer cushioning layer. The wound contacting layer may comprise a hydrocolloid adhesive with or without fibers embedded into the hydrocolloid.

### EXAMPLE 5

### Scrape Patch in Wound Care Applications

Similar multiple layer non-woven fabrics can be used in wound care application for hard to conform body areas such as knee, shoulder, elbow, and the bottom of the sole. The use of medication can be delivered to the areas along with a protective covering. The sprayed fibers may include typical textile fibers or synthetic polymers fibers currently being used in wound care applications.

### EXAMPLE 6

### External Packaging

In addition to the formulation examples as described above, the sprayable composition may be packaged into a pressurized, but propellant-free container for spraying (e.g. Bag-on-Valve, Bag-in-Can, Power Pouch, or Mixtek co-dispensing packaging technologies). A funnel device can be attached (permanently or not) to the spray head to provide a fixed spray area and spray distance for the output fibers. The multilayer compositions may be stored in one container with multiple compartments to accommodate the various formulations. The multilayer spray can be applied directly on skin or to form-fitting clothing to deliver the required therapeutic effect. The spray formulation may contain additional surfactants (such as polysorbates, cationic metal fatty esters) to enhance the dispersion and solubilization of the fibers.

### EXAMPLE 7

### Wound Contact Layer Sprayable Film

A 13% w/w of (53/8/39) TRIS(3-methacryloyloxypropyltris(trimethylsiloxy)silane/isooctylacrylate/methyl methacrylate terpolymer in 50:50 solvent combination of isooctane/hexamethyldisiloxane solvent is sprayed on the wound to form a film. A fiber containing spray is sprayed over the film.

We claim:

1. A spray-on, non-woven fabric system suitable for reducing or alleviating a discomforting condition of the skin and/or an area underlying the skin when applied thereto, comprising:
a first spray-on composition comprising a material suitable for reducing or alleviating a discomforting condition of the skin and/or an area underlying the skin when applied thereto; and
a second spray-on composition, different from the first spray-on composition, for covering the first spray-on composition when applied thereto, the second composition comprising fibers, a binder for the fibers and a volatile carrier for solubilizing the binder in the second composition.

2. The system according to claim 1 wherein the material is selected from the group consisting of a source of heat, a source of cooling, an antibiotic, an anesthetic, an anti-inflammatory, and an anti-itch composition.

4. The system according to claim 2 wherein the source of heating is selected from the group consisting of iron oxide powder, capsaicin, zinc-copper and other redox systems.

4. The system according to claim 2 wherein the source of cooling is selected from the group consisting of menthol, camphor and hydrogels.

5. The system according to claim 1 further comprising a third spray-on composition.

6. The system according to claim 5 wherein the third spray-on compositions comprises a source of heat or cooling.

7. The system according to claim 1 wherein the first spray-on composition is selected from the group consisting of a hydrocolloid adhesive, a hydrogel and an ointments.

8. The system according to claim 7 wherein the hydrocolloid adhesive is selected from the group consisting of a hydrophobic adhesive selected from the group consisting of silicones, polyisobutylenes, acrylics and natural rubbers, and a fluid absorbing material selected from the group consisting of methylcellulose, hydroxyethyl methylcellulose, hydroxypropyl methylcellulose, ethylhydroxyethyl cellulose, hydroxyethylcellulose, hydroxypropylcellulose, carboxymethlcellulose, polyvinyl alcohol, polyvinylpyrrolidone, polyethylene oxide, gum arabic, gum karaya, gum tragacanth, guar gum, gum benzoin, alginic acid and salts of any of the above.

9. The system according to claim 1 wherein the fibers are polymeric and have a length from 0.02 to 0.15 mm, the binder is selected from the group consisting of styrene butadiene copolymers, styrene isoprene block copolymers, hydrogenated butadiene styrene copolymers, hydrogenated isoprene styrene block copolymers, polyvinyl acetate, polyvinyl butyrate, natural latex and polyvinyl alcohol, and the volatile carrier is selected from the group consisting of a C₂ to C₁₂ ether, a C₂ to C₁₂ alcohol, a C₂ to C₁₂ ketone, a C₅ to C₁₂ hydrocarbon and a C₂ to C₁₂ ester.

10. A multilayer, non-woven fabric topical covering for reducing or alleviating a discomforting condition of the skin and/or an area underlying the skin when applied thereto, comprising:
a first layer comprising a material suitable for reducing or alleviating a discomforting condition of the skin and/or an area underlying the skin; and
a second layer, different from the first layer, covering the first layer and comprising fibers and a binder for the fibers.

11. The topical covering according to claim 10 wherein the material is selected from the group consisting of a source of heat, a source of cooling, an antibiotic, an anesthetic, an anti-inflammatory and an anti-itch composition

12. The topical covering of claim 11 wherein the source of heat is selected from the group consisting of iron oxide powder, capsaicin, zinc-copper and other redox systems.

13. The topical covering according to claim 11 wherein the source of cooling is selected from the group consisting of menthol, camphor and hydrogels.

14. The topical covering according to claim 10 further comprising a third composition.

15. The topical covering according to claim 14 wherein the third layer comprises a source of heat or cooling.

16. The topical covering according to claim 10 wherein the first layer is selected from the group consisting of a hydrocolloid adhesive, a hydrogel and an ointment.

17. The wound covering according to claim 16 wherein the hydrocolloid adhesive is selected from the group consisting of a hydrophobic adhesive selected from the group consisting of silicones, polyisobutylenes, acrylics and natural rubbers, and a fluid absorbing material selected from the group consisting of methylcellulose, hydroxyethyl methylcellulose, hydroxypropyl methylcellulose, ethylhydroxyethyl cellulose, hydroxyethylcellulose, hydroxypropylcellulose, carboxymethlcellulose, polyvinyl alcohol, polyvinylpyrrolidone, polyethylene oxide, gum arabic, gum karaya, gum tragacanth, guar gum, gum benzoin, alginic acid and salts of any of the above.

18. The wound covering according to claim 10 wherein the fibers are polymeric and have a length from 0.02 to 0.15 mm, the binder is selected from the group consisting of styrene butadiene copolymers, styrene isoprene block copolymers, hydrogenated butadiene styrene copolymers, hydrogenated isoprene styrene block copolymers, polyvinyl acetate, polyvinyl butyrate, natural latex and polyvinyl alcohol, and the volatile carrier is selected from the group consisting of a C₂ to C₁₂ ether, a C₂ to C₁₂ alcohol, a C₂ to C₁₂ ketone, a C₅ to C₁₂ hydrocarbon and a C₂ to C₁₂ ester.
